(19) United States
(12) Patent Application Publication
(10) Pub. No.: US 2022/0395207 A1
(43) Pub. Date: Dec. 15, 2022

(54) THERMAL IMAGING DIAGNOSTIC METHOD AND SYSTEM THEREOF

(71) Applicant: THERMANOSTICS LTD., Jerusalem (IL)

(72) Inventor: Alona Goldstein, Jerusalem (IL)

(21) Appl. No.: 17/753,778

(22) PCT Filed: Sep. 16, 2020
(86) PCT No.: PCT/IL2020/051013

§ 371 Date: Mar. 14, 2022

Related U.S. Application Data
(60) Provisional application No. 62/900,667, filed on Sep. 16, 2019.

(51) Int. Cl. A61B 5/24; A61B 5/00

(52) U.S. Cl. CPC A61B 5/24; A61B 5/0022; G16H 40/63

(57) ABSTRACT

The present invention discloses an imaging diagnostic method and system thereof. More particularly, the present invention discloses a method which takes thermal, infrared-based high-resolution images of a hand or a foot of a mammalian subject, and generates a map of neurogenic spots, indicative of different medical conditions or abnormalities in specific organs. Additionally, the system of the present invention can provide recommendations for further testing, future treatments, or an immediate treatment on the neurogenic spots.

### FIELD OF THE INVENTION

The present disclosure relates to a medical diagnostic method and system. More particularly, the present disclosure relates to a thermal, infrared-based high-resolution imaging of the hand or the foot providing diagnostic indications of organs which might be out of balance or developing medical problems based on neurogenic spots.

### BACKGROUND OF THE INVENTION

As the global population is rapidly growing, the average life span is dramatically increasing and epidemies such as diabetes, cancer and cardiovascular diseases are becoming more common, more and more people are in need of medical services. Along with the increasing morbidity rates, the awareness of early detection of diseases, the availability of medical treatments and the demand for adequate and affordable medical services are rising. In addition, many medical conditions are not easy to detect and patients usually have to undergo a plurality of tests, which are expensive, time-consuming, require a highly trained and skilled staff, might take a long time to interpret and could involve invasive procedures, pain or discomfort. Furthermore, some diseases lack technical methods for early diagnosis and in some cases, the patients see their doctors only when their condition is far too progressive or unmanageable. Taken together, these factors take an enormous financial toll on the medical system worldwide, which spends approximately 75% of its resources and budgets on chronic illnesses. As a result, the present medical system is still lagging behind in terms of meeting the patient's needs and solutions for these problems are required.

Nonetheless, in recent years the medical field has advanced, mainly due to global trends which are revolutionizing its basic core by shifting the spotlight from treating chronic illnesses to personalized medicine, preventive medicine, early detection of medical conditions, and maintaining wellness rather than focusing on illness.

One of the main fields mostly impacted by these changes is the detection of diseases in early stages. Currently, the most commonly used tests to detect biomarkers (powerful and useful indicators telling whether a system or an organ is off equilibrium) are blood, urine or stool tests mainly preformed at hospitals and clinics, on the order of a physician. As stated above, these tests are not always focused and they burden the system both financially and administratively. As a direct result, the market of diagnostic tools is constantly improving.

The fast-growing industry of POC (point of care diagnostic) aims to simplify and minimize medical test equipment, thus rendering it accessible to as many people as possible and additionally, practicable outside of medical settings at the patient's convenience, time and location of interest. The most popular devices are designed to detect blood levels of glucose and glycated hemoglobin, coagulation, fertility problems, infectious diseases, cardiac pathologies, thyroid activity etc.

Moreover, wearable sensors which monitor vital signs, such as pulse, respiratory rate, temperature and oxygen saturation and the currently under development all-in-one systems which include at-home kits, mobile applications and portable labs are also becoming leading forces in shaping the face of modern and future medicine.

Additionally, important developments take place in the imaging field. Much effort is made in improving imaging devices, especially shifting from using the deleterious, potentially carcinogenic radiation employed for example in CT scans to using non-ionizing radiation, such as infrared in thermography.

U.S. Pat. No. 8,792,968B2 to Song Xiao et al. from Sep. 27, 2014 discloses a thermal imaging device to be employed for the evaluation of medical conditions. That device is designed to use non-hazardous radiation, to take images of different zones of the human body and to produce thermal gradient sectional views from the thermal image representing conditions such as peripheral blood microcirculation, metabolism, psychological, muscle endurance and more. Moreover, the computer program of the device references from a patients' database.

Patent application WO2018002862A1 to Krithika Venkataramani from Jan. 4, 2018 discloses a system and a method for classifying the hormone receptor status of malignant tumorous tissue identified in a thermographic image of a human breast.

US patent 2009216130(A1) to Raphael Hirsch et al. from Jun. 8, 2010 discloses a method for quantifying and assessing arthritis, swelling, heat distribution, erythema, and range of motion in the joints using among other modalities thermal imaging and visible and near-infrared imaging. This invention also includes a handprint mold to stabilize the imaged hand in a standard pose, so scans are obtained from two viewpoints (left and right).

US patent 2009326381(A1) to Yun-e Yuan et al. from Dec. 31, 2009 discloses a system and a method for evaluating health-related conditions in a human subject using thermal imaging. This invention relates to the identification of different parts of the human body based on bone structures and establishing different viscera and bowels, vessels and known meridians of the human subject based on the geometric ratios between the bone structures and parts of the human subject.

Patent application WO2018158504(A1) to Jouni Ihme from Sep. 7, 2018 discloses a system for diagnosing the existence and progression of diseases by thermal imaging and mapping temperatures of human body parts, and analyzing differences between human body parts and changes in view of time.

US patent 2016271167(A1) to Shui Yin Lo from Sep. 22, 2016 discloses a method for detection, treatment, and prevention of neurological development disorders utilizing an infrared imaging device, meridian theory of traditional Chinese medicine, and predetermined amounts of solid water particles to detect, treat, and prevent a neurological developmental disorder, such as autism.

Francis Ring reviews in his publication “Thermal Imaging Today and Its Relevance to Diabetes” (Journal of Diabetes Science and Technology, volume 4, issue 4, July 2010) the history of clinical thermal imaging technique and discloses the use of the latter to diagnose diabetes by measuring the temperature of the periphery of the limbs (hands and feet). However, this report solely refers to the detection of diabetes and does not relate to other disorders or to the performance of other organs, nor does it correlate between localized spots found in the hand or foot to different organs.

In view of the prior art and given the various challenges described above, there is still an unmet long-felt need to develop an easy-to-use diagnostic device that can readily detect and diagnose a vast range of medical conditions at the early stages in a relatively short time, at the point of care, without preforming lab tests and in a non-invasive, pain-free manner, using high-resolution infrared thermal imaging.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

FIG. 1 depicting a schematic presentation of the thermal imaging examination procedure of the present invention;

FIG. 2 depicting a flowchart of the method for image analysis and comparison to other databases performed by the software of the present invention;

FIG. 3 depicting two different possible configurations of the thermal imaging device of the present invention;

FIG. 4 depicting a graphical presentation of possible positions of the hand that is imaged in the present invention;

FIG. 5 depicting analyzed thermal images obtained from the procedure of the present invention.

FIG. 6 depicting a flowchart of the diagnosis and treatment processes of the present invention;

FIG. 7 depicting a schematic presentation of block chain of the present invention; and

FIG. 8 depicting a schematic presentation of a preferred embodiment of the present invention.

### SUMMARY OF THE INVENTION

It is an object of the present invention to disclose a method of assessing the health state and wellness of a mammalian subject, comprising steps of:
a. operating a high-resolution infrared thermal imaging system placed in adjacent to a hand or a foot of said mammalian subject for collecting thermal images of said hand or foot; and
b. obtaining an image or a set of images of said hand or foot with neurogenic spots for diagnostic purposes.

It is another object of the present invention to disclose a method of assessing the health state and wellness of a mammalian subject, comprising steps of:
a. obtaining a thermal image or a set of images of a hand or a foot of said mammalian subject comprising neurogenic spots;
b. operating a data processor for (i) optimizing said images of said hand or foot, (ii) defining said neurogenic spots and (iii) constructing a coordinates' map of said neurogenic spots;
c. creating a database of neurogenic spots patterns that correlate with different diagnosed abnormalities serving as pre-defined maps.
d. operating an algorithm for (i) comparing said coordinates' map to pre-defined database maps, (ii) matching pattern of said coordinates' map to said pre-defined databases maps, (iii) associating between said coordinates' map to organs or abnormalities, (v) storing said coordinates' map in said database;
e. generating a report based on the findings of said images.

It is another object of the present invention to disclose the method above, wherein said thermal images are taken by any thermal imaging device, further wherein steps b-e are carried out remotely.

It is another object of the present invention to disclose the method above, wherein said method further generating a list of recommendations based on the findings of said images, said recommendations are selected from a group consisting of: taking additional medical tests, taking additional thermal images, consulting a medical professional, changing habits, changing a lifestyle, changing a diet, initiating a treatment and any combination thereof.

It is another object of the present invention to disclose the method above, wherein said method further generating a treatment protocol.

It is another object of the present invention to disclose the method above, wherein said treatment is a stimulation of said neurogenic spots, selected from a group consisting of chemical, electrical, biological or physical stimulation by contact or non-contact manner.

It is another object of the present invention to disclose the method above, wherein said treatment is selected from a group consisting of: pharmacological treatment, surgical intervention, bio-feedback, rehabilitative treatment, magnetic stimulation, wellness-related treatments, alternative treatments, acupuncture, massage, dietary treatments, physiotherapy, exercise, and any combination thereof.

It is another object of the present invention to disclose the method above, wherein said organs are selected from a group consisting of: brain, eyes, ears, nasal cavity, tongue, back, throat, gums, teeth, pharynx, larynx, esophagus, stomach, pancreas, liver, gallbladder, small intestine, large intestine, bile ducts, anus, rectum, liver, kidneys, spleen, thyroid, duodenum, appendix, bones, joints, knees, elbows, heart, trachea, lungs, bronchi, diaphragm, spinal cord, urinary bladder, ureters, urethra, adrenal gland, salivary glands, mammary glands, pituitary gland, tonsils, prostate, testes, penis, uterus, ovaries, fallopian tubes, nerves, thymus, lymph nodes, parotid glands, ligaments, muscles, integumentary system, tendons and any combination thereof.

It is another object of the present invention to disclose the method above, wherein said abnormalities are selected from a group consisting of: pathologies, infections, inflammations, obstructions, neoplasm , necrosis, dysplasia, hypertrophy, hyperplasia, hypoplasia, organomegaly, aneurysm, thromboembolism, diabetes, fibromyalgia, migraine, neurological disorders, immunological disorders, hematological disorders, endocrine disorders, autoimmune disorders, metabolic disorders, neurodegenerative disorders, respiratory disorders, genetic disorders, ergonomic and musculoskeletal disorders, rheumatic disorders, fibrotic diseases, dermatological diseases, cardiovascular diseases, nutritional deficiencies, lesions, stress-related manifestations and any combination thereof.

It is another object of the present invention to disclose the method above, wherein said mammalian subject is selected from a group consisting of human, feline, canine, cercopithecine, elephantine, simian, leonine, lupine, murine, ursine, and vulpine.

It is another object of the present invention to disclose the method above, wherein said hand or foot or any part thereof is photographable from the frontal side, back side, side view or from 360° in view in at least one posture, further wherein said hand or foot or any part thereof is imaged at least once, more preferably several consecutive times to generate a video.

It is another object of the present invention to disclose the method above, wherein said hand and foot are left hand or foot, right hand or foot, or any combination thereof.

It is another object of the present invention to disclose the method above, wherein said neurogenic spots are hot spots, cold spots and any combination thereof.

It is another object of the present invention to disclose the method above, wherein said data processor is configured to decode said images of said hand or foot of said mammalian subject and to associate said images to patterns of abnormalities utilizing computational functions, selected from a group consisting of: big data analysis, artificial intelligence, machine vision, machine learning algorithms and any combination thereof.

It is another object of the present invention to disclose the method above, wherein said pre-defined databases maps are maps collected from databases selected from a group consisting of: maps comprising images collected by any imaging device, maps comprising images stored in said database, statistically decoded maps (a map associated with a certain abnormality/condition which was generated by the system of the present invention based on the deciphering of numerous similar images presenting similar patterns of neurogenic spots), publicly available maps of eastern methodologies, maps known in the art, previous thermal images of said hand or foot of said mammalian subject or other mammalian subjects and any combination thereof.

It is another object of the present invention to disclose the method above, wherein said system is an adaptive self-learning system configured to update said pre-defined databases once obtaining new images and generate new analyses.

It is another object of the present invention to disclose a map of neurogenic spots according to any one of claims 1-16, wherein the pattern of said neurogenic spots is indicative to a status of said mammalian subject, selected from a group consisting of an abnormality, well-being, basal healthy state and any combination thereof.

It is another object of the present invention to disclose a system for assessing the health state and wellness of a mammalian subject, said system comprising:
a. a high-resolution infrared thermal imaging device configured to collect thermal images from the hand or foot of said mammalian subject, said device further comprising:
i. a thermal detector;
ii. a lens; and
b. a data processor configured to (i) optimize said images of said foot or hand, (ii) define neurogenic spots and (iii) construct a coordinates' map of said neurogenic spots; and
c. an algorithm configured to (i) compare said map to pre-defined database maps, (ii) match pattern of said coordinates' map to said pre-defined databases maps, (iii) associate between said coordinates' map to organs or abnormalities, (v) store said coordinates' map in said database;
   wherein said system is configured to generate a report based on the findings of said images, further wherein said system is configured to provide a treatment for said mammalian subject at the point of care at any time point.

It is another object of the present invention to disclose the system above, further comprising an apparatus selected from the group consisting of: fixture for said hand or foot, a background screen, a chamber for temperature stabilization, a case or a kit to reduce external disturbances or noises, components for in situ stimulation of said neurogenic spots and any combination thereof.

It is another object of the present invention to disclose the system above, wherein said organs are selected from a group consisting of: brain, eyes, ears, nasal cavity, tongue, back, throat, gums, teeth, pharynx, larynx, esophagus, stomach, pancreas, liver, gallbladder, small intestine, large intestine, bile ducts, anus, rectum, liver, kidneys, spleen, thyroid, duodenum, appendix, bones, joints, knees, elbows, heart, trachea, lungs, bronchi, diaphragm, spinal cord, urinary bladder, ureters, urethra, adrenal gland, salivary glands, mammary glands, pituitary gland, tonsils, prostate, testes, penis, uterus, ovaries, fallopian tubes, nerves, thymus, lymph nodes, parotid glands, ligaments, muscles, integumentary system, tendons and any combination thereof.

It is another object of the present invention to disclose the system above, wherein said abnormalities are selected from a group consisting of: pathologies, infections, inflammations, obstructions, neoplasm (benign or malignant tumors), necrosis, dysplasia, hypertrophy, hyperplasia, hypoplasia, organomegaly, aneurysm, thromboembolism, diabetes, fibromyalgia, migraine, neurological disorders, immunological disorders, hematological disorders, endocrine disorders, autoimmune disorders, metabolic disorders, neurodegenerative disorders, respiratory disorders, genetic disorders, ergonomic and musculoskeletal disorders, rheumatic disorders, fibrotic diseases, dermatological diseases, cardiovascular diseases, nutritional deficiencies, lesions, stress-related manifestations and any combination thereof.

It is another object of the present invention to disclose the system above, wherein said mammalian subject is selected from a group consisting of human, feline, canine, cercopithecine, elephantine, simian, leonine, lupine, murine, ursine, and vulpine.

It is another object of the present invention to disclose the system above, wherein said treatment is stimulation of said neurogenic spots, selected from a group consisting of chemical, electrical, biological or physical stimulation by contact or non-contact manner.

It is another object of the present invention to disclose the system above, wherein said high resolution is at least 400×240 pixels.

It is another object of the present invention to disclose the system above, wherein said thermal detector is configured to have a thermal sensitivity smaller than 100 mK.

It is another object of the present invention to disclose the system above, wherein said thermal detector is configured to have a thermal sensitivity smaller than 50 mK.

It is another object of the present invention to disclose the system above, wherein said thermal detector is within the infra-red wavelength range, said range is selected from a group consisting of: long wavelength infrared of 7.5-14 μm, medium wavelength infrared of 3-5 μm, short wave infrared of 0.7-2.5 μm and any combination thereof.

It is another object of the present invention to disclose the system above, wherein said thermal detector is optionally configured to have a manual or automatic zoom system.

It is another object of the present invention to disclose the system above, wherein said hand or foot or any part thereof is photographable from the frontal side, back side, side view or from 360° in view at least one posture at least once, more preferably several consecutive times to generate a video.

It is another object of the present invention to disclose the system above, wherein said hand and foot are left hand or foot, right hand or foot, or any combination thereof.

It is another object of the present invention to disclose the system above, wherein said lens is configured to have a wide angular opening, suitable for short distance imaging.

It is another object of the present invention to disclose the system above, wherein said angular opening is preferably between 15-90°.

It is another object of the present invention to disclose the system above, wherein said background screen is a surface placed behind said hand or foot.

It is another object of the present invention to disclose the system above, wherein said chamber for temperature stabilization is configured to have a controlled temperature therein to stabilize and acclimate the skin temperature of said hand or foot before imaging.

It is another object of the present invention to disclose the system above, wherein said components for in situ stimulation of said neurogenic spots are located in said chamber or in another chamber and are configured to apply external stress on said neurogenic spots by means selected from a group consisting of: chemical, electrical, physical, biological, irradiation, heat, cool and any combination thereof.

It is another object of the present invention to disclose the system above, wherein said data processor is configured to decode said images of said hand or foot of said mammalian subject and to associate said images to patterns of abnormalities utilizing computational functions, selected from a group consisting of: big data analysis, artificial intelligence, machine vision, machine learning algorithms and any combination thereof.

It is another object of the present invention to disclose the system above, wherein said pre-defined databases maps are maps collected from databases selected from a group consisting of: maps comprising images collected by any imaging device, maps comprising images stored in said database, publicly available maps of eastern methodologies, maps known in the art, previous thermal images of said hand or foot of said mammalian subject or other mammalian subjects and any combination thereof.

It is another object of the present invention to disclose the system above, wherein said system is an adaptive self-learning system configured to update said pre-defined databases once obtaining new images and generate new analyses.

It is another object of the present invention to disclose the system above, wherein said data processor is configured to interpret said pre-defined database maps and to statistically calculate the likelihood of suffering from said abnormality.

It is another object of the present invention to disclose the system above, wherein said system is mobile or stationary.

It is another object of the present invention to disclose the system and method above, wherein said system is configured to be operable via a mobile phone as a mobile application.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a method for detecting, diagnosing and treating organs' abnormalities in mammalian subjects using high resolution infrared thermal imaging system of the hand or the foot.

The term “abnormalities” generally refers hereinafter to any imbalance or medical problem manifested in an organ, a tissue or a system. This can be any sort of pathological conditions, infections (including bacterial, viral, fungal and parasitic infections), inflammations, obstructions, neoplasm (benign or malignant tumors), necrosis, dysplasia, hypertrophy, hyperplasia, hypoplasia, organomegaly, aneurysm, thromboembolism, diabetes, fibromyalgia, migraine, neurological disorders, immunological disorders, hematological disorders, endocrine disorders, autoimmune disorders, metabolic disorders, neurodegenerative disorders, respiratory disorders, genetic disorders, ergonomic and musculoskeletal disorders, rheumatic disorders, fibrotic diseases, dermatological diseases, cardiovascular diseases, nutritional deficiencies, lesions, stress-related manifestations, such as headaches, chronic or idiopathic pain, digestive issues, rapid heartbeat, sweating and the like.

As used herein after, the term “neurogenic spot” refers to localized foci of spots found under the skin, that can be either cold spots or hot spots, depending on their intensity and temperature.

The hot spots for instance are characterized by a high temperature, increased sensitivity, high electrical conductivity, vasodilation, extravasation of plasma and a size of about 0.2-4 mm. The neurogenic hot spots are formed when an organ experiences stress and sends signals to the brain through the nervous system. These signals are also transmitted to the skin due to the branching of the nerves. At certain points, the neurons induce the release of neuropeptides into the adjacent tissue and evoke localized neurogenic inflammation. Hence, these spots reflect irregularities manifesting in the organs.

As used herein after, the term “map” refers to the output received from the imaging system of the present invention or from other imaging devices or systems. The map can be for example a thermal image of the subject's hand or foot showing neurogenic spots at different intensity levels in different locations of the hand/foot. That correlates systematically with different pathologies and/or disorders.

As used herein after, the term “wellness” refers to the well-being and quality of life of a subject. More specifically, this term embodies the notions of preventing diseases, prolonging life and optimizing life quality.

As used herein after, the term “thermography” refers to the field of diagnosing medical conditions based on thermal imaging.

As used herein after, the term “about” refers to any value being up to 25% lower or greater the defined measure.

The present invention, named thermanostics, provides a new method of thermography, based on the appearance of small neurogenic spots in the imaged hand or foot, indicating different medical conditions or abnormalities in specific organs/system.

Chronic diseases start developing in the body a long time before they are even noticeable, let alone treated. This happens as an organ experiences some sort of stress and gets off balance. Since the organs are connected to the brain through the nervous system, signal transmission between the brain and the affected organ suggesting a pathological condition, can be monitored to detect abnormalities in the early stages of development. The above-mentioned signal transmission results in inflammatory neurogenic spots under the skin. The patterns of the neurogenic spots can be unique, signifying an individual's physical state or they can appear in several different subjects, showing the same abnormality.

The present invention discloses a method for detecting neurogenic spots in the hand or the foot of a mammalian subject, using a high resolution infrared thermal imaging system and for associating these neurogenic spots to different organs and abnormalities. The software of the system analyzes, processes and optimizes the resultant images and compares them to other databases and references using machine learning algorithms, machine vision and artificial intelligence to detect a specific abnormality in an organ, based on corresponding neurogenic spots. In addition, the present invention discloses a method to thermally image patients and to produce a report specifying medical conditions and treatment solutions and protocols based on images of neurogenic spots. The system can also provide treatment to the patients while being examined or shortly afterward.

Compared to other thermal imaging techniques known in the art, the main differences and advantages of the present invention are:
a. The body part that is imaged is the hand or the foot and not the entire body or big areas such as the breasts, rendering the imaging procedure more comfortable to the patient and more practicable to perform at a location and time of interest.
b. The imaging procedure is significantly shorter than other known practices, as only a small body part is examined.
c. The present invention provides high resolution and sensitivity, as the detected neurogenic spots are small-sized (only few millimeters) in relative to bigger body parts disclosed in the prior art.
d. The detected neurogenic spots of the present invention are not limited to acupoints along known meridians. The spots can be found anywhere along any nerve, especially in the ends of the limbs, where tens of thousands of sensory neurons endings are located, representing the highest neuronal density in the body. Therefore, each spot in the hand can be a neuro-point and get inflamed as a result of a stress somewhere else in the body, that is connected to this nerve ending.
e. The present invention utilizes a designated software harnessing big data analysis, machine learning, machine vision and artificial intelligence algorithms to link between each unique neurogenic spot to a specific organ and/or an abnormality. This is done by collecting data from patients manifesting similar abnormalities, identification of common patterns of neurogenic spots, creating specific databases and comparing the data to the specific databases generated by the system of the present invention or to publicly known references, such as maps of Chinese/Indian/Korean methodologies or other maps of traditional medicine.
f. Treatment of neurogenic spots in the hand or foot can be performed as a new stand-alone therapy even in cases where no known reference map is found, to cause a beneficial effect and contribute to the well-being of the subject.
g. The treatment can be done “offline”, meaning marking the locations of the neurogenic spots and then apply treatment by a therapist or by the patient himself/herself, or “online”, meaning a device locating the neurogenic spots and automatically treat them at the point of care (mechanically or by other means of stimulation).
h. The detection of the neurogenic spots is indicative of diseases and medical conditions even in the early stages of development. The sooner this examination is preformed and results are obtained, the sooner a treatment can be initiated.

In a preferred embodiment of the present invention, the learning and identification processes of representative neurogenic spot maps for a certain abnormality may be performed by a direct study of neurogenic spots distribution observed in patients with a specific abnormality, i.e. finding a common pattern of neurogenic spots, by means of data analysis and machine learning, in a population that was previously diagnosed by conventional means, or in correlation with the results of other diagnostic methods, such as blood test, ultrasound, x-ray scans etc. or by correlating the neurogenic spots to maps known in alternative medicine techniques, such as acupuncture, reflexology and su-jok. Different abnormalities will result in different maps. For example, a neurogenic spot map related to breast cancer will be different than a map related to diabetes. The level of confidence for a certain diagnosis can be set by the statistical analysis of big data of maps related to patients versus control. The intensity of the neurogenic spot may be a measure of the severity of the related abnormality.

In yet another preferred embodiment of the present invention, a single map can be indicative of several different medical conditions and/or abnormalities. The method and system of the present invention can match and superimpose a plurality of maps to detect and decipher a plurality of abnormalities. For instance, a specific pattern of neurogenic spots below the fingernails can indicate a problem in the spleen, whereas another specific pattern of neurogenic spots at the center of the palm in the same image is associated with abnormalities of the lungs. The system is designed to operate in such a fashion that it can detect both patterns of neurogenic spots presented in a single image, compare between various maps stored in its databases, and associate them with different abnormalities.

In yet another preferred embodiment of the present invention, the entire imaging process takes only a few minutes and it can be performed outside of hospitals and clinics, for instance, at the patient's house.

In another preferred embodiment of the present invention, the system and method generate a medical report, specifying potential abnormalities and treatment solutions.

In another preferred embodiment of the present invention, the system and method also provide therapeutic means to treat a patient during or shortly after the imaging process, based on the findings concerning the neurogenic spots. The treatment can include inter alia applying pressure on the neurogenic spots, irradiating them with different wavelengths or stimulating them mechanically, electrically, biologically, chemically or by heat or cool, so signals are transmitted from the neurogenic spots back to the brain to induce healing processes. Such evidence of therapeutic effects was demonstrated in rats, where electrical or manual stimulation of neurogenic inflammatory spots alleviated the symptoms of the associated organs (development of hypertension or colitis) in rats, which was likely due to the release of endogenous opioids (see Kim D H., Kim H. Y. et al, “Acupuncture points can be identified as cutaneous neurogenic inflammatory spots”, Scientific Reports, 7: 15214, November, 2017).

In yet another preferred embodiment of the present invention, the examination is carried out in a temperature-controlled environment with prefixed temperature and humidity conditions.

In yet another preferred embodiment of the present invention, the method and system also provide a small chamber for temperature stabilization and thermal acclimation prior to the imaging process. Fifteen minutes usually suffice to at least achieve a reasonable level of stability in blood pressure and skin temperature. During this equilibration period, the hands or feet to be imaged must be unclothed. Additionally, the system of the present invention comprises a device or a kit or a housing for reducing or dissipating external noises or environmental disturbances, for instance, a device which reduces infrared irradiation emitted from other sources in proximity to the system.

In yet another preferred embodiment of the present invention the method can provide a general status of the physical condition and/or wellness of the patient.

In yet another preferred embodiment of the present invention, the method and system can monitor changes observed over time, or after treatment, by following the changes between periodic thermal images and thus evaluate the patient's condition and state.

In another preferred embodiment of the present invention, the method can be applied to other medical or clinical diagnostic techniques, such as optical coherence tomography (OCT), electrical conductivity/impedance mapping, chemical staining for inflammation bio-markers and the like.

In yet another preferred embodiment of the present invention, the system collects details about the subject before, during or after the examination to better evaluate its condition. The details can be inter alia the subject's gender, weight, height, medical history, habits and more.

In yet another preferred embodiment of the present invention, the method and system can thermally detect both neurogenic hot spots, meaning spots whose intensity is higher in relative to their surroundings, and neurogenic cold spots, meaning spots whose intensity is lower in relative to their surroundings. The two type of spots can be indicative of medical problems.

In yet another preferred embodiment of the present invention, the operation of the system in question and the evaluation of the results obtained from the imaging processes can be carried out remotely, by means of telemedicine. In other words, the patient undergoes the imaging procedure at his/her house or in a clinic or a hospital, whereas the doctor/technician/healthcare professionals are found in another location and are able to evaluate, diagnose or treat the patient at a distance using telecommunication technologies. Furthermore, in additional preferred embodiment of the present invention, the patient can download a designated mobile application to his/her own mobile phone, possibly conduct the test using the phone's camera communicate with the healthcare professional and receive the results to the phone.

In yet another preferred embodiment of the present invention, the analysis of the results obtained from the present system or other similar imaging systems is carried out by means of artificial intelligence, and not by human professionals. The results of the imaging process, the map showing the neurogenic spots, recommendations for further diagnostic tests, instructions for self-treatment (for instance, applying pressure on the neurogenic spots by using electrical, mechanical or radiation means) or other recommendations for treatments given by professional providers (such as pharmacological treatment, surgical intervention, bio-feedback, rehabilitative treatment, magnetic stimulation, wellness-related treatments, alternative treatments, acupuncture, massage, dietary treatments, physiotherapy, exercise etc.) can be received via the mobile application from either human professionals or from AI-based means.

### EXAMPLE 1

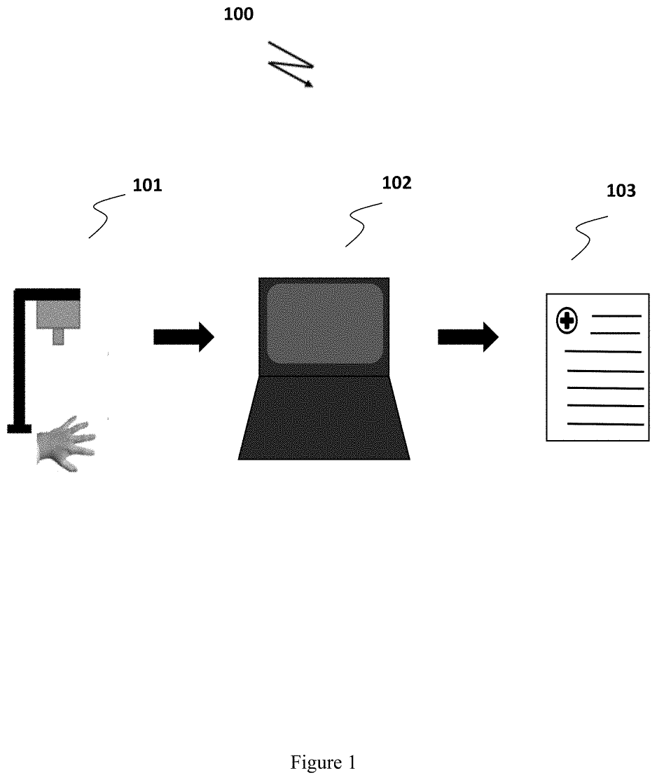

Reference in now made to FIG. 1 depicting a schematic presentation of the thermal imaging examination procedure 100 of a preferred embodiment of the present invention. A patient arrives at a location where the imaging device is set up, then the patient's hand is photographed by the thermal imaging device (101). After obtaining an image or a set of images and analyzing it by a designated software installed on a computer or a remote server/cloud (102), a diagnostic report is generated (103), specifying potential organs which might be manifesting abnormalities. Furthermore, recommendations are made as well, which might include sending the patient to further examinations or advising the patient to consult with a doctor or to adopt a healthier life style (quit smoking, exercise more regularly, change diet etc.)

### EXAMPLE 2

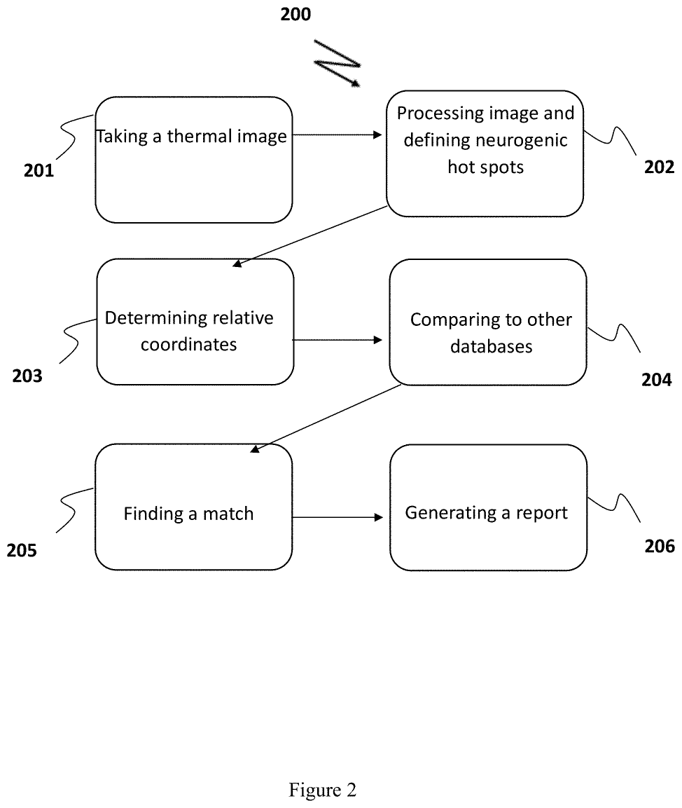

Reference is now made to FIG. 2 depicting a flowchart 200 of the method for image analysis of the acquired images, and comparison to other databases performed by the software of the present invention. During the examination a thermal image of the patient's foot or hand is taken (201). Subsequently, a designated software processes, analyzes and optimizes the images to detect neurogenic spots representing an abnormality in an organ (202). Then, relative coordinates of the neurogenic spots are determined using the software (203) and the spots are compared against other databases of pre-defined unique maps related to different health conditions (204). In the following step a match is found in other databases, so the detected neurogenic spot can be associated with a specific organ/medical condition (205). At the end of the process, a report is generated based on the above data and analysis specifying potential health problems and treatment solutions and protocols is also produced (206).

### EXAMPLE 3

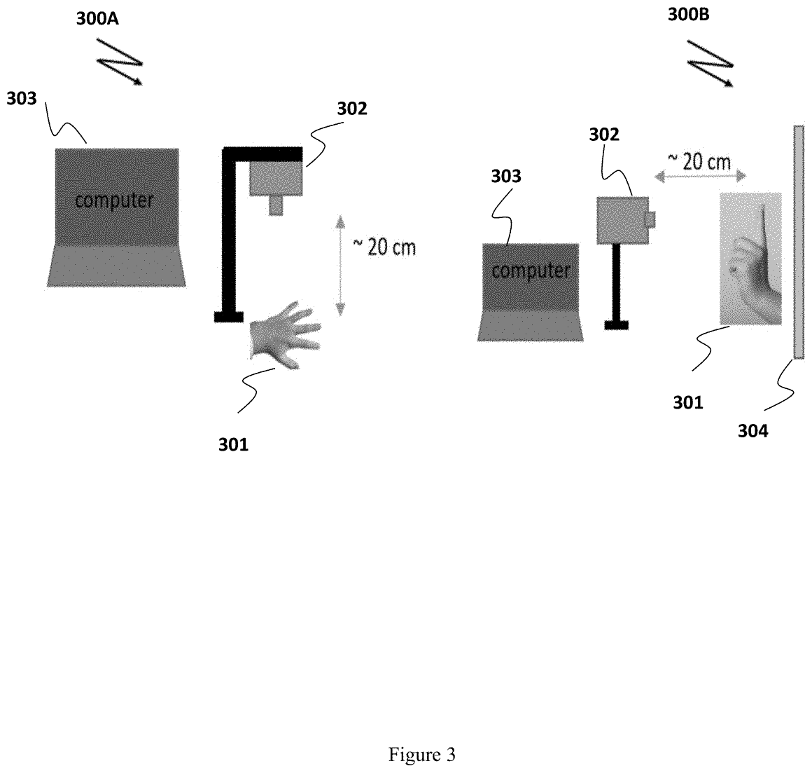

The imaging of the hand or the foot can be performed from various positions and directions. Reference in now made to FIG. 3 depicting two different possible configurations of the thermal imaging device of the present invention. On the left side of the figure 300A, the patient's examined hand (301) is positioned approximately 20 cm below the thermal imaging device (302) and it is facing down. The device (302) is connected to a computer (303), in which the designated software analyzing and interpreting the detected neurogenic spots is installed. On the right side of the figure 300B, the patient's examined hand (301) is positioned approximately 20 cm in front of the thermal imaging device (302), where only one finger is imaged. The device (302) is connected to a computer (303), in which the designated software analyzing and interpreting the detected neurogenic spots is installed. Optionally, a background screen (304) is positioned behind the examined hand (301).

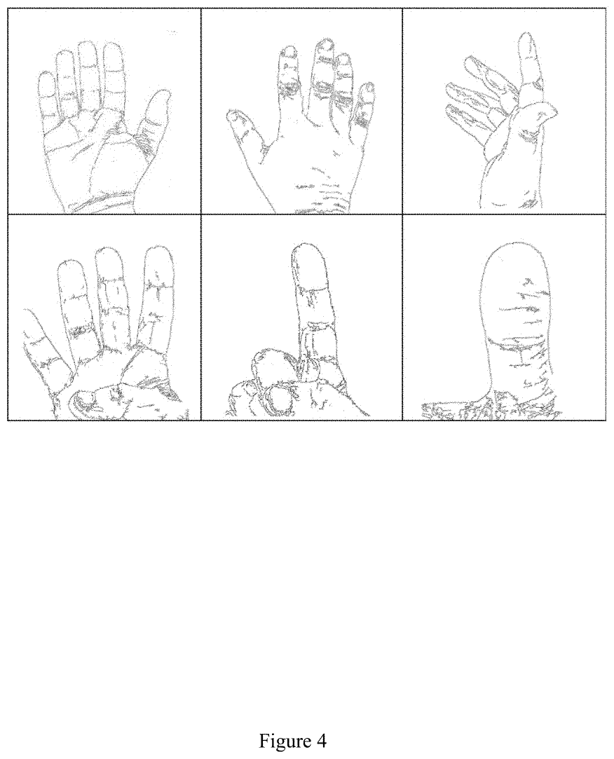

Reference in now made to FIG. 4, where more possible postures of the patient's hand are presented. The thermal imaging can be performed in various positions: one can collect frontal images of the hand where either the palm or the back of the hand is facing the imaging device, side view images of the hand, images of one or several fingers and more. In addition, one image can be taken or several images to create a short video.

In another non-limiting example of the present invention, the hand or the foot can be imaged by a plurality of cameras, with different combination of wavelengths, not particularly in the infrared spectrum, that simultaneously take images from different directions or by a rotating camera that circles the hand or foot and captures images from different angles or generate a video comprising of various images.

In yet another non-limiting example of the present invention, images can be taken from either the left or the right hand or foot, or from both. This combination and comparison between the two hands/foots could provide more information and accuracy to the subject's health evaluation.

### EXAMPLE 4

When taking high-resolution thermal images, differences in the location and appearance of the neurogenic spots can be observed within different subjects or within the same subject over the course of time.

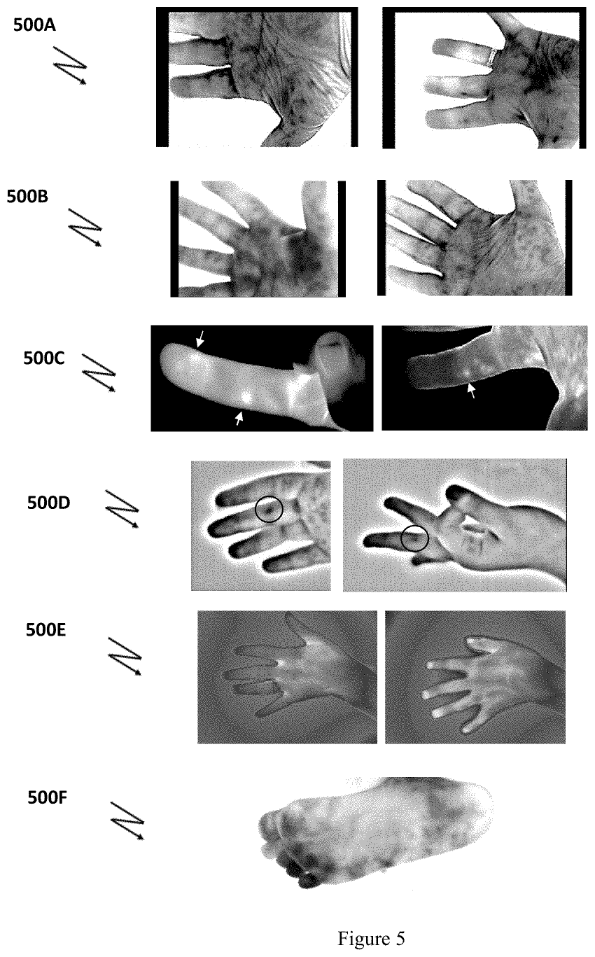

Reference in now made to FIG. 5 showing images obtained from the thermal imaging system of the present invention after being processed and optimized by the software. This method can compare for example, between different human subjects, as it can be seen in 500A. Significant differences are observed between the left image, where the neurogenic spots tend to spread in the middle parts of the palm, especially below the fingers, compared to the right image, where the spots are more intense and tend to cluster in the center and the lower right part of the palm.

In 500B the palm of the same human subject is examined over a course of 30 days. In the left image, which was the first one to be taken, there is a conspicuous concentration of neurogenic spots below the thumb, which seems to fade away in the right image, taken after a month. When the first image was taken, the subject was suffering from pneumonia and this part of the palm is associated with the lungs, according to some eastern methodologies.

In 500C, the finger of another human subject is imaged over a course of 30 days. In both images the same spot appears in the middle of the finger. In the image on the left, another neurogenic spot appears in the upper part of the finger. These two specific spots are associated with the liver according to the su-jok maps. Blood tests of this subject revealed that her blood sugar levels are higher than the standard values and that she is anemic. Both of these symptoms are abnormalities affecting and related to the liver.

In 500D thermal images of a subject's palm are presented. In both images hot neurogenic spots can be observed in the middle of one of the fingers (circled and marked black). According to su-joks maps and to additional maps generated by the system of the present invention, this specific location of a neurogenic spot is associated with the right and left knees. After undergoing medical tests, it turns out that the subject has indeed inflammation in both knees.

500E depicts an example of thermal images of the backside of a palm. The left image was taken when the subject was not experiencing any unusual medical condition, and therefore, indicating the basal state, whereas in the right image, neurogenic spots are observed as white clusters in the fingernails, a location which is associated with headaches, according to some eastern methodologies.

In 500F a thermal image of a foot is presented showing a representative pattern of hot neurogenic spots (shown in black) associated with abnormalities in the stomach.

To associate between the acquired images and the different organs and abnormalities, the present invention utilizes a technology that employs algorithms of big data, machine learning, machine vision and artificial intelligence. These algorithms identify the location of the neurogenic spots from the resulting thermal images and compare them to pre-defined maps to find matches. Moreover, as the system is a learning system, these images are stored and serve as a reference for future patients with the same medical conditions.

### EXAMPLE 5

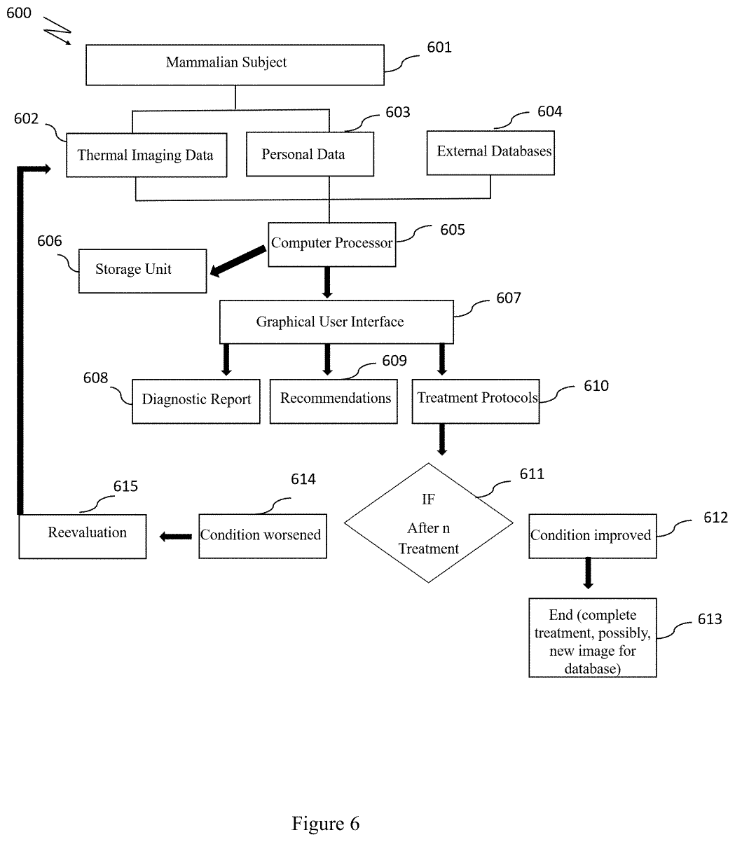

Reference is now made to FIG. 6, where flowchart of the diagnosis and treatment processes of the present invention (600) is depicted. A mammalian subject (601) arrives at the point of care, where the system of the present invention is located. The system collects thermal images of the subject's hand or foot and personal details, and stores them as thermal imaging data (602) and personal data (603) respectively. In addition, the system contains external databases (604), which comprise data obtained from other patients and decoded maps, achieved by Big Data analysis or by the comparison to known maps according to different eastern methodologies. The system's software (computer processor; 605) processes and optimizes the resultant images to detect a pattern of neurogenic spots. Then, the computer processor (605) utilizes all the above databases, and cross-references between them to find maps with neurogenic spots resembling the map obtained from the mammalian subject (601). Once a match is found, the processor (605) employs known databases and links between the location of the neurogenic spots to organs and medical abnormalities using special algorithms of artificial intelligence and data analysis. The processor (605) also stores all the data obtained from the mammalian subject (601) in the storage unit (606) for future purposes (comparing the condition of the same subject over the course of time or entering the collected images to the external databases to be compared and referenced against other subjects' images). Subsequently, the processor (605) uses a graphical user interface (607) to generate a diagnostic report (608), a list of recommendations (609) and treatment protocols (610). All of the above are also stored in the storage unit (606). The subject (601) or its therapist can decide which treatment it prefers to undergo based on the diagnostic report (608). After a number of treatments (referred to in the figure as “n”), the subject (601) faces several options (611); if the treatment is successful and its condition improves (612), it can either complete the treatment or stop it and take additional thermal images (613) of its hand or foot to be stored in the storage unit (606) and in the external databases (604) for reference. However, if the treatment is not effective and the condition of the mammalian subject (601) gets worse (614), the therapist can re-evaluate the situation (615) and have the subject (601) repeat the procedure (600) to see if the new image is different from previous ones, if new matches from the external databases (604) can be found or if new treatment protocols (610) can be followed. Alternatively, the therapist can advise the subject to stop the treatment completely and to seek other treatment options.

### EXAMPLE 6

A block chain is a system in which a growing list of records of digital transactions are maintained across several computers that are linked in a peer-to-peer network. The blockchain operates by spreading a continuous growing ledger of records, which are time stamped over a wide array of private computers across the world. This assures that the records cannot be altered, modified, deleted or tampered with.

Block chain is designed specifically to accelerate and simplify the process of how transactions are recorded. This means that any type of asset can be transparently transacted using this completely decentralized system.

The present invention is a system which is also suitable for block chain ledger.

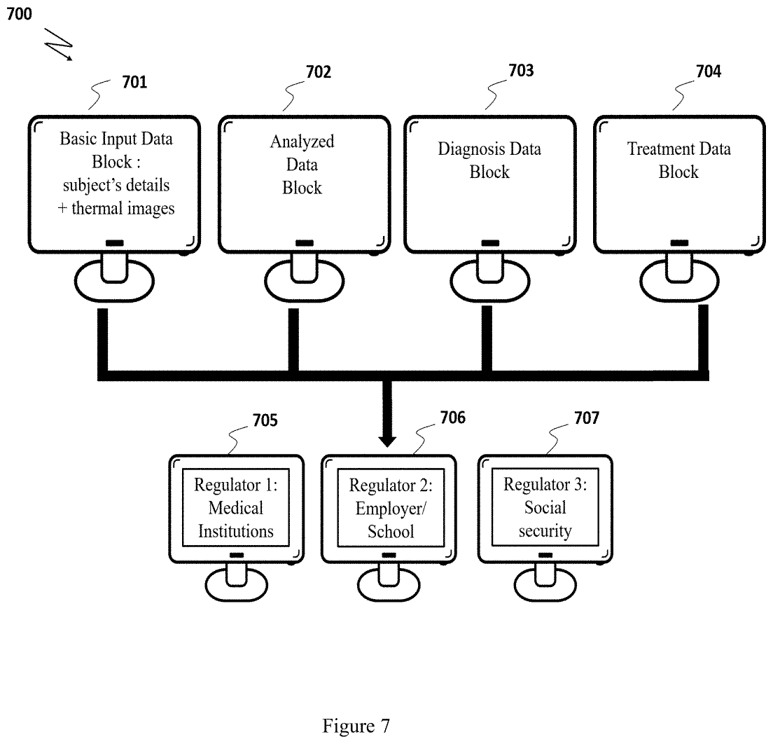

Reference is now made to FIG. 7, where the block chain of the present invention is illustrated (700). The data obtained from the examination of the present invention is collected in several different blocks (list of records): the personal details of the subject (basic input data block; 701), analyzed data block (702) which includes data about the processed images, the pattern of neurogenic spots and their matches found in other databases, the diagnosis data block (703) which contains data about the associations between the locations of the spots to organs and abnormalities and the treatment data block (704), containing data about suggested treatment protocols and recommendations concerning lifestyle and daily habits. All this data can be collected and securely transferred to other relevant and interested parties' databases, such as medical institutions (705), workplaces or schools (706) or to social security or other welfare services (707).

### EXAMPLE 7

Machine learning can be described as a software that changes, when it learns new information. As the software is self-adaptive, it is not necessary to manually add new rules. Deep learning is the most prospective area of machine learning. It is based on neural networks that require large volumes of data to teach themselves. While most of the data is stored in hard copy form, the current trend is aiming at rapid digitization of these large amounts of data. These massive quantities of data (known as ‘big data’) hold the promise of supporting a wide range of functions, including in the medical and healthcare fields, such as clinical decision support, disease surveillance, and population health management.

The big data of the present invention is collected by the thermal images obtained from each examination combined with the personal and medical information of each subject, and is compared to data obtained from other sources: other users of the present invention, other databases and known maps of spots from eastern methodologies. According to a preferred embodiment, the present invention is a machine learning system, which teaches itself to compare between neurogenic spots of one patient to spots from other databases and to generate based on these comparisons, a diagnostic report with potential treatment protocols and recommendations to the patient.

### EXAMPLE 8

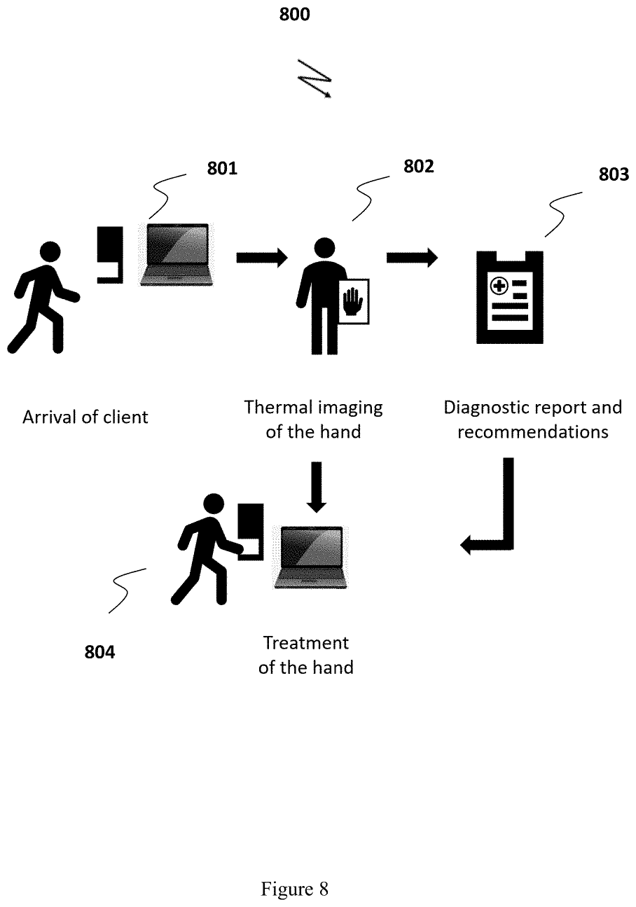

Reference in now made to FIG. 8 depicting a schematic presentation of the thermal imaging examination procedure 800 combined with the possibility of receiving a treatment at the point of care by the system of the present invention. A patient arrives at a location where the imaging device is set up (801), then the patient's hand is photographed by the thermal imaging device (802). After obtaining an image or a set of images and analyzing it by the software, a diagnostic report is generated (803), specifying potential organs which might be manifesting abnormalities. Furthermore, recommendations are made as well, which might include sending the patient to further examinations or undergoing an immediate treatment at the point of care (804). The treatment can be carried out after the diagnosis report is issued or immediately after the imaging and detection of the neurogenic spots. In a non-limiting example, the system employs local irradiation to stimulate the detected neurogenic spots in a non-contact manner.

### EXAMPLE 9

Reference is now made to non-limiting examples of a potential use of the present invention:

Mila is a 36-year old female patient, who has been unwell for a while. Mila arrives to the point of care, where the system of the present invention is stationed. Before the procedure starts, Mila is asked to provide some details and to fill a short questionnaire on a computer software concerning her age, gender, physical state, medical history, quotidian routine, number of children and more. As it is winter and the temperature outside is 15° C., Mila is asked to place her hand in a small chamber, where the temperature and humidity are controlled, to allow the temperature of her hand to acclimate and stabilize. After Mila had her hand acclimated to the desired temperature, she places her hand on a designated fixture, where her palm is facing directly the device. The device collects several images of her hand, and then Mila is free to rest until the images are analyzed and interpreted and a report is produced. The designated software installed in the system of the present invention processes the images, optimizes them and detects two neurogenic spots, whose intensity is higher compared to their surroundings (i.e. hot spots). Then, the software compares the locations of these two specific spots to other images and maps of the human hand stored in its databases, using algorithms of big data, machine vision and artificial intelligence. After several matching images are found, the software associates the location of the spots to two organs: the liver and the pancreas. Concomitantly, the software saves Mila' s image following her consent, and stores it in the databases for future comparisons. Subsequently, the system generates a report for Mila, indicating that her liver and pancreas might be manifesting medical abnormalities. The report also utilizes statistics and states that Mila's specific combination and location of neurogenic spots may indicate X % probability of type 2 diabetes. In addition, Milla is recommended by the system to undergo several blood tests and to consult with her doctor. Mila can also opt to undergo a short treatment at the point of care, and if she chooses to do so, the therapist will stimulate the neurogenic spots by applying physical pressure, needling or electrical stimulation Alternatively, the system can issue instructions to Milla of how she can apply pressure on the spots by herself. Furthermore, Milla can choose a third option, in which the system uses local infrared irradiation for example, to stimulate the spots without any physical contact. These sorts of stimulations are believed to trigger the nervous system to transmit information to the brain that some organs are under stress, so the brain can start healing processes.

In another non-binding example of the present invention, George, a 42-year old male who is a heavy smoker, downloads a designated application to his own mobile phone, which is equipped with an advanced thermal camera. George takes a thermal image of his left hand using his mobile phone. Via the mobile application, George is able to take a number of actions: George can download a map of his hand, showing the neurogenic spots generated by the system of the present invention. George can also send the images to be analyzed and deciphered in a main server or a remote cloud and to obtain within minutes a specification of organs which might start to exhibit abnormal manifestations and/or abnormalities or medical conditions that he might be developing. In addition, George can download a list of recommendations provided to him by the system. Those recommendations can be for instance, to go and have blood and urine tests for liver enzymes, quit smoking and reduce sodium consumption. Moreover, George can be instructed by the application how to treat himself or alleviate his condition(s) by receiving explanations on which spots he should apply pressure to, the level of intensity that he should apply, and the duration and frequency of the sessions. All these information and instructions can be displayed to George in a short video clip generated by the application.

In another non-binding example of the present invention, Michael, a 68-year old male arrives to the ER at a hospital, complaining about pains and discomfort in his abdomen, fever and chills. Michael has several other comorbidities and has undergone a total hip arthroplasty recently. The ER doctors suspect that Michael might be manifesting a bacterial infection caused by Klebsiella pneumoniae, which can result in either pneumonia or urinary tract infection (UTI). To test the origin of the infection, the doctors take a thermal image of Michael's left palm using the present invention, and obtain a map showing a neurogenic spot pattern, which matches according to the system's analysis, to abnormalities of the urinary tracts. Based on the information received from the present invention, the doctors run an ultrasound scan and corroborate the system's analysis, that Michael suffers from UTI.

In an additional example for a use of the system of the present invention, Sheila, a 58-year old female, who was previously tested by the system and was shown to exhibit neurogenic spots on her right palm associated with breast cancer, goes back to the clinic, where the thermal imaging system is located, two year after the first image was taken. After her first visit, Sheila received a report advising her to consult her doctor and have a mammogram. Sheila was indeed diagnosed with breast cancer and had a surgery to remove the tumor. Sheila is currently healthy and would like to assess her medical status using the system of the present invention. Sheila's right palm is imaged, and Sheila is requested by the system to wait for several minutes until the analysis is completed. When Sheila goes back into the room, she is contacted by her doctor via means of telemedicine (her doctor is located in another city and speaks to her via a computer). The doctor who received from the system a map and an analysis comparing the current image to Sheila's previous image, reassures Sheila that her condition seems to be fine, and that the intense neurogenic spots associated with the breasts do not manifest in the current image.

In another example, physicians in the Covid-19 departments in Saint Claus hospital take thermal images of their patients' hands by any conventional thermographic means (any camera or device suitable for taking thermal infrared images of the human body). The physicians are interested in checking which patient might be developing pulmonary conditions, heart conditions or kidney failure which are too premature to detect by other conventional medical means. The physicians send the images on a daily basis to a remote cloud for further analysis. The patients' images are compared by the system of the present invention to various databases: images previously collected and analyzed by said system of the same patient or other patients, previous images collected by the physicians from the covid-19 department, publicly available maps of eastern methodologies and more. The system is configured to associate patterns of neurogenic spots belonging to the lungs/heart/kidneys/based on the above databases, and match them to the newly obtained images. The system sends the data to the physicians, who can now monitor more closely patient who might be at risk, and start giving them necessary preventive treatments. Additionally, the physicians update the system on the patients' medical status, and as a result, the system's databases are being updated correspondingly. In other words, if a map belonging to patient X shows neurogenic spot pattern associated with the kidneys, and patient X indeed suffers a kidney failure, patient X's map(s) is then added to a specific database which contains maps of kidney-associated neurogenic spot patterns. This picture can serve for future analysis, and whenever the system is introduced with a new image presenting neurogenic spots which might indicate a renal condition, the system will retrieve patient X's image(s), among other images, and compare them against the new image.

The system of the present invention can be installed in clinics and medical centers, and patients can get thermal images of their hands upon arrival, before seeing the doctors. For example, a patient takes a thermal image of her hand when she arrives to the clinic, and the doctor receives the resultant map and analysis thereof produced by the system and method of the present invention to her computer. The patient complains about back pain, which are shown in her map, but the system also indicates a pattern of neurogenic spots associated with the thyroid. The doctor asks the patient to undergo several tests, which show that the patient has hypothyroidism. The doctor prescribes the patient medications for this condition, and after a few weeks, the patient notices that she is less tired, and that symptoms such as constipation, weakness and dry skin from which she was suffering without being aware of their origin, are getting weaker and more tolerable.

The system and method of the present invention can be useful in rapid screening of large populations of people in places such as airports shopping centers, movie theaters etc. Currently, when all countries are concerned with a global pandemic (covid-19) and try to find solutions for better coping with it, the system and method of the present invention can be implemented in airports. As the disease is characterized by respiratory distress and pulmonary abnormalities, the system of the present invention can be installed as part of the security tests that passengers undergo, and each passenger would have thermal images of his/her hand taken and analyzed to test if the resultant map presents neurogenic spots associated with the lungs, respiratory track or respiratory syndromes. It is plausible that at first, the maps would indicate spots associated with general pulmonary or respiratory conditions, but once a large number of infected people are imaged and analyzed, a unique pattern of neurogenic spots specific for covid-19 would be generated.

Another possible usage of the present system and method is described in the following example: A subject arrives at a pharmacy, where an imaging station is installed. The system is designed to test nutritional deficiencies. The subject places his/her hand in the imaging system, and a thermal image is taken. The image is subsequently sent to a remote processor (cloud), which compares the resultant pattern of neurogenic spots to other maps stored in its database, presenting different nutritional deficiencies. Once a match has been found to the subject's map, the system generates a short report, recommending to the subject to start consuming vitamin D. Hence, the system can save the subject time in seeing doctors and waiting in lines.

The system of the present invention can be installed for example in a gym. Every six months, all the people who attend the gym, are requested to take a thermal image of their hand after a 15-min intense aerobic workout and an additional imaging after 5 minutes of rest. In light of the findings and the maps, the system can identify people with cardiac conditions or musculoskeletal disorders, and recommend to them to get checked more thoroughly by professionals.

1. A method of assessing the health state and wellness of a mammalian subject, comprising steps of:
a. operating a high-resolution infrared thermal imaging system placed in adjacent to a hand or a foot of said mammalian subject for collecting thermal images of said hand or foot; and
b. obtaining an image or a set of images of said hand or foot with neurogenic spots for diagnostic purposes.

2. A method of assessing the health state and wellness of a mammalian subject, comprising steps of:
a. obtaining a thermal image or a set of images of a hand or a foot of said mammalian subject comprising neurogenic spots;
b. operating a data processor for (i) optimizing said images of said hand or foot, (ii) defining said neurogenic spots and (iii) constructing a coordinates' map of said neurogenic spots;
c. creating a database of neurogenic spots patterns that correlate with different diagnosed abnormalities serving as pre-defined maps.
d. operating an algorithm for (i) comparing said coordinates' map to pre-defined database maps, (ii) matching pattern of said coordinates' map to said pre-defined databases maps, (iii) associating between said coordinates' map to organs or abnormalities, (v) storing said coordinates' map in said database;
e. generating a report based on the findings of said images.

3. The method of claim 2, wherein said thermal images are taken by any thermal imaging device, further wherein steps b-e are carried out remotely.

4. The method of claim 2, wherein said method further generating a list of recommendations based on the findings of said images, said recommendations are selected from a group consisting of: taking additional medical tests, taking additional thermal images, consulting a medical professional, changing habits, changing a lifestyle, changing a diet, initiating a treatment and any combination thereof.

5. The method of claim 2, wherein said method further generating a treatment protocol.

6. The method of claim 2, wherein said treatment is a stimulation of said neurogenic spots, selected from a group consisting of chemical, electrical, biological or physical stimulation by contact or non-contact manner.

7. The method of claim 5, wherein said treatment is selected from a group consisting of: pharmacological treatment, surgical intervention, bio-feedback, rehabilitative treatment, magnetic stimulation, wellness-related treatments, alternative treatments, acupuncture, massage, dietary treatments, physiotherapy, exercise, psychological therapy and any combination thereof.

8. The method of claim 2, wherein said organs are selected from a group consisting of: brain, eyes, ears, nasal cavity, tongue, back, throat, gums, teeth, pharynx, larynx, esophagus, stomach, pancreas, liver, gallbladder, small intestine, large intestine, bile ducts, anus, rectum, liver, kidneys, spleen, thyroid, duodenum, appendix, bones, joints, knees, elbows, heart, trachea, lungs, bronchi, diaphragm, spinal cord, urinary bladder, ureters, urethra, adrenal gland, salivary glands, mammary glands, pituitary gland, tonsils, prostate, testes, penis, uterus, ovaries, fallopian tubes, nerves, thymus, lymph nodes, parotid glands, ligaments, muscles, integumentary system, tendons and any combination thereof.

9. The method of claim 2, wherein said abnormalities are selected from a group consisting of: pathologies, infections, inflammations, obstructions, neoplasm , necrosis, dysplasia, hypertrophy, hyperplasia, hypoplasia, organomegaly, aneurysm, thromboembolism, diabetes, fibromyalgia, migraine, neurological disorders, immunological disorders, hematological disorders, endocrine disorders, autoimmune disorders, metabolic disorders, neurodegenerative disorders, respiratory disorders, genetic disorders, ergonomic and musculoskeletal disorders, rheumatic disorders, fibrotic diseases, dermatological diseases, cardiovascular diseases, nutritional deficiencies, lesions, stress-related manifestations and any combination thereof.

10. The method according to claim 1 or claim 2, wherein said mammalian subject is selected from a group consisting of human, feline, canine, cercopithecine, elephantine, simian, leonine, lupine, murine, ursine, and vulpine.

11. The method according to claim 1 or claim 2, wherein said hand or foot or any part thereof is photographable from the frontal side, back side, side view or from 360° in view in at least one posture, further wherein said hand or foot or any part thereof is imaged at least once, more preferably several consecutive times to generate a video.

12. The method according to claim 1 or claim 2, wherein said hand and foot are left hand or foot, right hand or foot, or any combination thereof.

13. The method according to claim 1 or claim 2, wherein said neurogenic spots are hot spots, cold spots and any combination thereof.

14. The method of claim 2, wherein said data processor is configured to decode said images of said hand or foot of said mammalian subject and to associate said images to patterns of abnormalities utilizing computational functions, selected from a group consisting of: big data analysis, artificial intelligence, machine vision, machine learning algorithms and any combination thereof.

15. The method of claim 2, wherein said pre-defined databases maps are maps collected from databases selected from a group consisting of: maps comprising images collected by any imaging device, maps comprising images stored in said database, statistically decoded maps, publicly available maps of eastern methodologies, maps known in the art, previous thermal images of said hand or foot of said mammalian subject or other mammalian subjects and any combination thereof.

16. The method of claim 2, wherein said system is an adaptive self-learning system configured to update said pre-defined databases once obtaining new images and generate new analyses.

17. A map of neurogenic spots according to any one of claims 1-16, wherein the pattern of said neurogenic spots is indicative to a status of said mammalian subject, selected from a group consisting of an abnormality, well-being, basal healthy state and any combination thereof.

18. A system for assessing the health state and wellness of a mammalian subject, said system comprising:
a. a high-resolution infrared thermal imaging device configured to collect thermal images from the hand or foot of said mammalian subject, said device further comprising:
i a thermal detector;
ii. a lens; and
b. a data processor configured to (i) optimize said images of said foot or hand, (ii) define neurogenic spots and (iii) construct a coordinates' map of said neurogenic spots; and
c. an algorithm configured to (i) compare said map to pre-defined database maps, (ii) match pattern of said coordinates' map to said pre-defined databases maps, (iii) associate between said coordinates' map to organs or abnormalities, (v) store said coordinates' map in said database;
wherein said system is configured to generate a report based on the findings of said images, further wherein said system is configured to provide a treatment for said mammalian subject at the point of care at any time point.

19. The system of claim 18, further comprising an apparatus selected from the group consisting of: fixture for said hand or foot, a background screen, a chamber for temperature stabilization, a device to reduce external disturbances or noises, components for in situ stimulation of said neurogenic spots and any combination thereof.

20. The system of claim 18, wherein said organs are selected from a group consisting of: brain, eyes, ears, nasal cavity, tongue, back, throat, gums, teeth, pharynx, larynx, esophagus, stomach, pancreas, liver, gallbladder, small intestine, large intestine, bile ducts, anus, rectum, liver, kidneys, spleen, thyroid, duodenum, appendix, bones, joints, knees, elbows, heart, trachea, lungs, bronchi, diaphragm, spinal cord, urinary bladder, ureters, urethra, adrenal gland, salivary glands, mammary glands, pituitary gland, tonsils, prostate, testes, penis, uterus, ovaries, fallopian tubes, nerves, thymus, lymph nodes, parotid glands, ligaments, muscles, integumentary system, tendons and any combination thereof.

21. The system of claim 18, wherein said abnormalities are selected from a group consisting of: pathologies, infections, inflammations, obstructions, neoplasm (benign or malignant tumors), necrosis, dysplasia, hypertrophy, hyperplasia, hypoplasia, organomegaly, aneurysm, thromboembolism, diabetes, fibromyalgia, migraine, neurological disorders, immunological disorders, hematological disorders, endocrine disorders, autoimmune disorders, metabolic disorders, neurodegenerative disorders, respiratory disorders, genetic disorders, ergonomic and musculoskeletal disorders, rheumatic disorders, fibrotic diseases, dermatological diseases, cardiovascular diseases, nutritional deficiencies, lesions, stress-related manifestations and any combination thereof.

22. The system of claim 18, wherein said mammalian subject is selected from a group consisting of human, feline, canine, cercopithecine, elephantine, simian, leonine, lupine, murine, ursine, and vulpine.

23. The system of claim 18, wherein said treatment is stimulation of said neurogenic spots, selected from a group consisting of chemical, electrical, biological or physical stimulation by contact or non-contact manner.

24. The system of claim 18, wherein said high resolution is at least 400×240 pixels.

25. The system of claim 18, wherein said thermal detector is configured to have a thermal sensitivity smaller than 100 mK.

26. The system of claim 18, wherein said thermal detector is configured to have a thermal sensitivity smaller than 50 mK.

27. The system of claim 18, wherein said thermal detector is within the infra-red wavelength range, said range is selected from a group consisting of: long wavelength infrared of 7.5-14 μm, medium wavelength infrared of 3-5 μm, short wave infrared of 0.7-2.5 μm and any combination thereof.

28. The system of claim 18, wherein said thermal detector is optionally configured to have a manual or automatic zoom system.

29. The system of claim 18, wherein said hand or foot or any part thereof is photographable from the frontal side, back side, side view or from 360° in view at least one posture at least once, more preferably several consecutive times to generate a video.

30. The system of claim 18, wherein said hand and foot are left hand or foot, right hand or foot, or any combination thereof.

31. The system of claim 18, wherein said lens is configured to have a wide angular opening, suitable for short distance imaging.

32. The system of claim 18, wherein said angular opening is preferably between 15-90°.

33. The system of claim 19, wherein said background screen is a surface placed behind said hand or foot.

34. The system of claim 19, wherein said chamber for temperature stabilization is configured to have a controlled temperature therein to stabilize and acclimate the skin temperature of said hand or foot before imaging.

35. The system of claim 19, wherein said components for in situ stimulation of said neurogenic spots are located in said chamber or in another chamber and are configured to apply external stress on said neurogenic spots by means selected from a group consisting of: chemical, electrical, physical, biological, irradiation, heat, cool and any combination thereof.

36. The system of claim 18, wherein said data processor is configured to decode said images of said hand or foot of said mammalian subject and to associate said images to patterns of abnormalities utilizing computational functions, selected from a group consisting of: big data analysis, artificial intelligence, machine vision, machine learning algorithms and any combination thereof.

37. The system of claim 18, wherein said pre-defined databases maps are maps collected from databases selected from a group consisting of: maps comprising images collected by any imaging device, maps comprising images stored in said database, statistically decoded maps, publicly available maps of eastern methodologies, maps known in the art, previous thermal images of said hand or foot of said mammalian subject or other mammalian subjects and any combination thereof.

38. The system of claim 18, wherein said system is an adaptive self-learning system configured to update said pre-defined databases once obtaining new images and generate new analyses.

39. The system of claim 18, wherein said data processor is configured to interpret said pre-defined database maps and to statistically calculate the likelihood of suffering from said abnormality.

40. The system of claim 18, wherein said system is mobile or stationary.

41. The system of claim 18 and the method of claim 2, wherein said system is configured to be operable via a mobile phone as a mobile application.
